(19) United States
(12) Patent Application Publication
(10) Pub. No.: US 2010/0063006 A1
(43) Pub. Date: Mar. 11, 2010

(54) COMPOSITIONS AND METHODS TO REDUCE FAT AND RETRACT SKIN

(75) Inventor: Diane I. Duncan, Fort Collins, CO (US)

Correspondence Address: CR MILES, P.C.;CRAIG R. MILES, 405 MASON COURT, SUITE 119, FORT COLLINS, CO, 80524, US

(73) Assignee: American Network of Lipolysis, LLC

(21) Appl. No.: 12/312,701

(22) PCT Filed: Nov. 21, 2007
(86) PCT No.: PCT/US07/24360

§ 371 Date: May 20, 2009

Related U.S. Application Data
(60) Provisional application No. 60/860,838, filed on Nov. 22, 2006.

(51) Int. Cl. A61K 31/661; A61P 3/00

(52) U.S. Cl. 514/121

(57) ABSTRACT

Compositions, methods, and apparatuses for treatment of subcutaneous fat tissue for the purpose of fat tissue reduction or other alterations of the subcutaneous fat tissue which affect the appearance of the overlying skin layer.

This application is the United States National Stage Application of International Patent Cooperation Treaty Application No. PCT/US2007/024360, filed Nov. 21, 2007, which claims the benefit of U.S. Provisional Patent Application No. 60/860,838, filed Nov. 22, 2006, each hereby incorporated by reference herein.

### I. TECHNICAL FIELD

The invention relates to compositions, methods, and apparatuses for treatment of subcutaneous fat tissue for the purpose of fat tissue reduction or other alterations of the subcutaneous fat tissue which affect the appearance of the overlying skin layer.

### II. BACKGROUND

In 1959, phosphatidylcholine (hereinafter “PC”) was isolated and used intravenously in Odessa, Russia, for the treatment of fat embolism. In 1988, Sergio Maggiore reported use of PC injections for cosmetic purposes. PC has also been used in treating xanthelasmas in Europe and in South America. In 1995, Dr. Patricia Rittes is believed to be the first to use subcutaneous injections of PC for the purpose of fat reduction. An injectable form of PC (LIPOSTABIL®, Sanofi-Aventis, Brigewater, N.J.) has been indicated for treatment of fat embolisms, coronary artery plaque, and fat tissue.

PC is often an ingredient in injectable fat reducing formulas. When isolated, it is produced as a powder. When reconstituted, it is quite viscous and must be mixed with a detergent, such as sodium deoxycholate (hereinafter “DC”), to solubilize it sufficiently to create an injectable form. DC is a bile salt that can function to make the PC soluble in water or other biocompatible solvents; otherwise, the PC can precipitate out of solution. DC has been described as having a “detergent” effect on fat dissolution in a porcine in vitro study and has nonspecific effects on both adipose and muscle cells. Other pharmaceuticals, such as Fungizone (Bristol Myers Squibb, New York, N.Y.) (an injectable form of amphotericin B), are commonly combined with bile salts to enhance their solubility and make them compatible with intravenous delivery.

Conventional formulation of DC without PC (“DC formulations”) and conventional formulations of PC combined with DC (“PC/DC formulations”) have been shown to achieve a level of fat reduction by in vivo histopathological studies. See for example FIG. 1 which is a image of a thin tissue section of an area of fat tissue one day post injection with a DC formulation of about 42 milligrams per milliliter. Macrophages (arrow A) dominate thin tissue section with prominent deposition of hyaline ground substance (arrow B) each an indication of profound fat necrosis.

However, even though conventional DC formulations and PC/DC formulations have been shown to achieve a level of fat reduction, substantial unresolved problems remain with the use of such conventional formulations and conventional methods of use.

A first significant problem with conventional DC formulations may be a marked prolonged inflammatory reaction along with excess fibrosis and collagen formation post injection. A strong histamine release may result within five to ten minutes post injection of DC formulations. Onset of swelling may be observed within thirty minutes of injection and appears to be a dose related reaction. Burning and pain following injection of DC formulations may persist for a month or more. Referring primarily to FIG. 2, which is a image of a thin tissue section at the dermal-fat junction one day post injection with DC 4.2% (DC 42 milligrams per milliliter). Extreme inflammation can be observed around the hair follicle (arrow C), eccrine sweat glands (arrow D), and the blood vessels (arrow E). Lysed fat cells are indicated by arrow F and remaining viable fat cell by arrow G.

A second significant problem with the use of conventional DC formulations and certain PC/DC formulations may be the failure to disperse sufficiently through fat tissue to avoid localized cavitation about the sites of injection or avoid areas of untreated uncavitated fat tissue between the injection sites. The failure of such conventional DC formulations or conventional PC/DC formulations to disperse between injection sites can result in an uneven layer of fat tissue supporting the skin layer which can feel or have an appearance of unevenness or lack of uniformity.

Now referring primarily to FIG. 3, a 4.2% DC formulation including 0.2 cubic centimeter (“cc”) methylene blue per 10 cc of the DC formulation administered 0.5 c per injection site at a depth of 10 millimeters (“mm”) with injection sites 1.5 centimeters (“cm”) apart can result in a pattern of localized dispersion of the DC proximate to the injection sites (dark colored areas-indicated by pointers) interrupted by areas in which the DC formulation has not dispersed (light colored areas) in tissue specimens harvested between about 15 minutes and even after one hour post injection.

One approach to generating a greater level of dispersion has been to utilize formulations of PC 5.0%-DC 4.2%. However, PC can cause cholinergic side effects such as nausea, vomiting, diarrhea, flushing, sweating, bradycardia, and the like when the total administered amount of PC exceeds about 2000 mg per treatment. The use of PC 5.0%-DC 4.2% in the context of the dose restriction may severely limit the size of the tissue region treated in a single session. Additionally, PC/DC formulations such as PC 4.0%-DC 4.2%; and PC 3.75%-DC 4.2% can each produce undesirable levels inflammation and necrosis of the adnexae of the venules, arterioles, sweat glands hair follicles and nerve elements. Now referring to FIG. 4, which is a image of a thin section obtained at the junction of the dermis and subcutaneous fat one week post injection with PC 4.0%-DC 4.2% shows substantial inflammation of the adnexae of the hair follicles (see arrow H), the eccrine sweat glands (see arrow I) and nerve elements (see arrow J). Referring now to FIG. 5, which is a image of a thin section of the skin elements one week post injection of the underlying fat layer with PC 4.0%-DC 4.2% shows that patchy necrosis of the sweat glands (arrow K) and blood vessels (arrow L) can occur by use of certain conventional formulations of PC/DC. Now referring to FIG. 6, which is a image of a thin section through the thickness of the dermis three weeks post injection of the underlying fat layer with PC 3.75%-DC 4.2% resulting in an area of necrobiosis which extends to about forty percent of the thickness of the deep dermis (see dashed line and arrow M pointing into area of necrobiosis left of the dashed line) and as shown by FIG. 7 which provides an image of a thin section of tissue at the dermal-fat junction can result in an extreme thickening of the fibrous septae (arrow N).

A third significant problem with the use of conventional DC and PC/DC formulations may be that the concentration of DC in certain formulations may not allow for optimum lipolytic activity. This may be a result of decreasing the amount of DC in DC only or PC/DC formulations to avoid inflammation, necrosis or necrobiosis of tissues into which DC disperses (whether due to the actual amount of DC utilized in a particular formulation or due to dilution of a particular formulation which results in a lower concentration of DC). Regardless, of the manner by which reduction of DC is achieved as to any formulation, amounts of DC of less than about twenty milligrams per milliliter can show substantial decrease in lipolytic activity.

A fourth significant problem with conventional PC/DC formulations can be that conventional ratios or concentrations of PC to DC may not be optimal. Prior to the discovery of the inventive formulations described herein, it is believed that it was not known and that the conventional teachings regarding DC formulations and PC/DC formulations did not disclose (whether expressly or inherently) the strong effect which the ratio of PC to DC in a formulation can have on the level of dispersion of the PC/DC formulation in subcutaneous fat tissue or the level of lipolytic action of the PC/DC formulations on subcutaneous fat tissue, or did not teach the ratios of particular PC/DC compositions described herein (or certain concentrations of PC/DC in a biocompatible solvent which provide such PC to DC ratios), or did not teach that the specific ratios or concentrations of the PC/DC compositions described herein had a greater efficacy with respect to dispersion or lipolysis in subcutaneous fat tissue.

The inventive PC/DC compositions and methods of using such PC/DC compositions described herein address each of the long felt but unresolved problems with the use of conventional DC formulations and conventional PC/DC formulations for the treatment of subcutaneous fat tissue above described, and provide PC/DC compositions having concentrations of PC and DC and PC to DC ratios which exhibit a increased level of dispersion and lipolytic action in subcutaneous fat tissue.

### III. DISCLOSURE OF INVENTION

Accordingly, a broad object of the invention can be to provide PC/DC compositions providing certain ratios of PC to DC weight to weight (wt/wt), or certain ratios of PC to DC wt/wt at certain concentrations, in a biocompatible solvent which can be administered by injection into subcutaneous fat tissue, and which can have one or more advantages of increased lipolytic activity, reduced inflammatory response, or increased dispersion characteristics, whether separately, collectively or in various permutations and combinations, as compared to conventional DC formulations or conventional PC/DC formulations.

Another broad object of the invention can be to provide compositions for injection having specific ratios of PC to DC for fat reduction which can provide increased lipolytic activity, a reduced inflammatory response, or increased dispersion characteristics which can further include an amount of benzyl alcohol, rapivacaine, isoproterenol hydrochloride (“ISUPREL™”); collagenase, such as Clostridial collagenase; or an amount of one or more of: nicotinic acid, clofibrate, tannic acid, scorpion toxin, snake venom, beta adrenergic stimulants, dimethlyaminoethanol, hyaluronic acid, penta-O-galloyl-alpha-D-glucose, hormone sensitive lipase, human adipose triglyceride lipase, tnf-alpha, raspberry ketone, ethanol, rosiglitazone, peroxisome-proliferator activated receptor gamma, Y-9738 (ethyl 2(4-chlorophenyl)-5-ethoxy-4-oxazoleacetate) oliphen, fish oil, scallop shell extract, peanut shell extract, and caffeine, separately or in various permutations and combinations.

Another broad object of the invention can be to provide methods of utilizing the inventive PC/DC compositions including without limitation utilizing an injection location identification template which can engage the surface of the skin overlaying the subcutaneous tissue to be injectably treated to identify the location of the plurality of injection locations on the surface of the skin (also referred to as a “skin layer surface”).

Naturally, further objects of the invention are disclosed throughout other areas of the specification, drawings, photographs, and claims.

### IV. BRIEF DESCRIPTION OF THE DRAWINGS

FIG. 1 is an image of a thin section of fat tissue about 24 hours post injection with a conventional DC formulation of DC 4.2% in saline.

FIG. 2 is an image of a thin section of tissue at the dermal-fat junction about 24 hours post injection with a conventional DC formulation of DC 4.2% in saline.

FIG. 3 is an image of a pattern of localized dispersion of a conventional DC formulation proximate to injection sites (dark colored areas) interrupted by areas in which the DC formulation has not dispersed (light colored areas) in tissue specimens harvested between 15 minutes and one hour post injection of 0.5 milliliter (“mL”) of 4.2% DC in saline at each of a plurality of injection sites about 1.5 cm apart.

FIG. 4 is an image of a thin section obtained at the junction of the dermis and subcutaneous fat one week post injection with a conventional PC/DC formulation PC 4.0%-DC 4.2% in saline.

FIG. 5 is an image of a thin section of the skin elements one week post injection of the underlying fat layer with a conventional PC/DC formulation PC 4.0%-DC 4.2% in saline.

FIG. 6 is an image of a thin section through the thickness of the dermis three weeks post injection of the underlying fat layer with a conventional PC/DC formulation PC 3.75%-DC 4.2% in saline.

FIG. 7 an image of a thin section of tissue at the dermal-fat junction one week post injection with a conventional PC/DC formulation PC3.75%-DC4.2% in saline.

FIG. 8 is a graph which summarizes data relating to the lipolytic action of various DC only formulations, PC/DC formulations, and substances which can be added to DC only formulations or PC/DC formulations.

FIG. 9 is an image of a pattern of substantially uninterrupted dispersion of a particular embodiment of an inventive PC/DC composition in tissue specimens harvested between 15 minutes and one hour post injection of 0.5 mL of a particular embodiment of the inventive PC/DC compositions (PC 2.66% (26.6 mg/mL)/DC 2.48% (24.8 mg/mL) in saline) at each of a plurality of injection locations about 1.5 cm apart.

FIG. 10 is a plan view of an embodiment of an injection location identification template.

FIG. 11 is a side view of an embodiment of an injection location identification template.

FIG. 12 is an end view of an embodiment of an injection location identification template.

FIG. 13 is a drawing which shows a person exhibiting marks viewable upon disengaging the injection location identification template from the surface of the skin each mark identifying a corresponding h injection location at which an amount of a PC/DC composition can be injectably delivered to the subcutaneous fat layer.

FIG. 14 is a schematic drawing which shows particular embodiments of an inventive method of injecting PC/DC composition into subcutaneous fat tissue for lipolysis.

FIG. 15 is a schematic drawing which shows a particular embodiment of an inventive method of injecting PC/DC compositions into subcutaneous tissue for treatment of cellulite.

FIG. 15 is a schematic drawing which shows a particular embodiment of an inventive method of injecting PC/DC compositions into subcutaneous tissue to affect skin retraction.

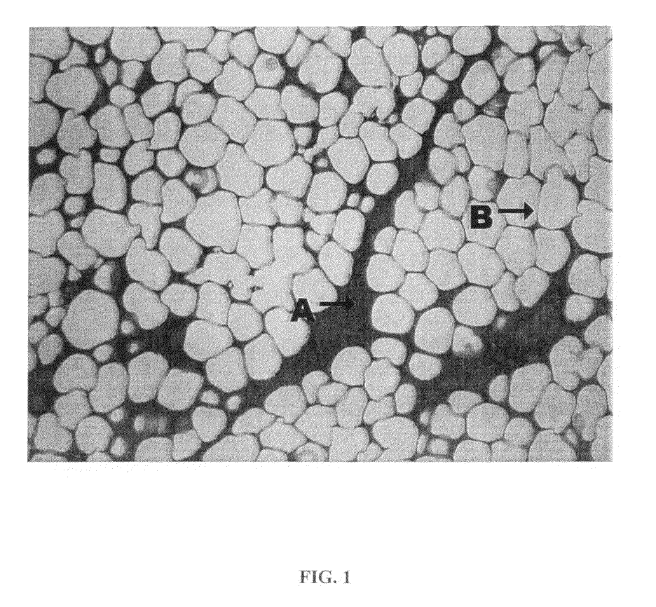

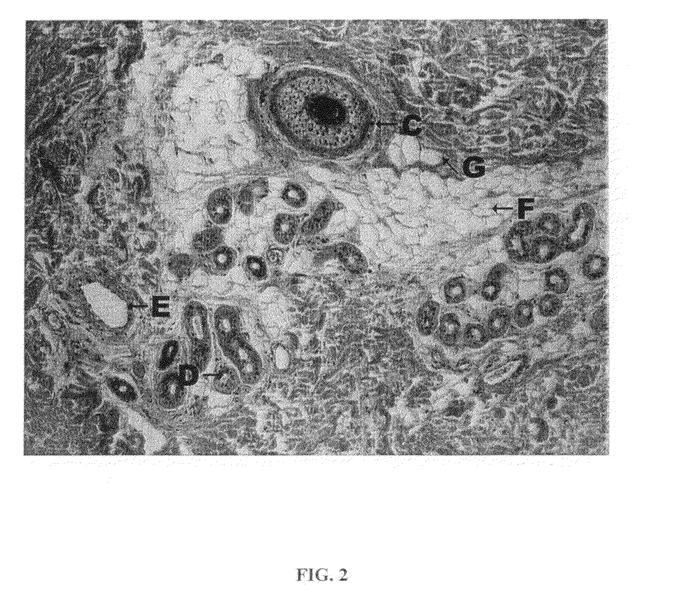

### V. MODE(S) FOR CARRYING OUT THE INVENTION

Generally, injectable compositions effective in reducing subcutaneous fat which provide an amount of phosphatidylcholine and an amount of deoxycholate in a ratio of between about 1.0:0.80 (wt/wt) and about 1.0:1.0 (wt/wt) in an amount of biocompatible solvent. Specifically, injectable compositions effective in reducing fat which provide an amount of phosphatidylcholine and an amount of deoxycholate in a ratio (PC:DC ratio) of between about 1.0:0.83 (wt/wt) and about 1.0:0.97 (wt/wt) in an amount of biocompatible solvent.

A range of efficacious embodiments of a PC/DC composition for fat reduction and skin retraction can be prepared by combining an amount of PC with an amount of DC in an amount of a biocompatible solvent to generate an solution having PC:DC ratios of between about 1.0:0.80 and about 1.0:1.0 (wt/wt).

The biocompatible solvent selected can be any material, without limitation and by way of example, any one of: an amount of water, an amount of saline (typically an isotonic solution of sodium chloride and distilled water), an amount of water combined with an amount of alcohol (typically added as a preservative), an amount of saline combined with an amount of alcohol, an amount of water combined with an amount of benzyl alcohol, an amount of saline combined with an amount of benzyl alcohol, or the like. The use of the term “biocompatible solvent” is not intended to preclude the interaction of PC with DC as a factor in allowing PC or DC or both to be solubilized in the amount of biocompatible solvent.

Certain embodiments of the inventive PC/DC composition can provide a solution which provides an amount of PC of between about 24.0 milligrams per milliliter and about 32.0 milligrams per milliliter of the biocompatible solvent and further provides an amount of DC of between about 19.2 milligrams per milliliter and about 32.0 milligrams per milliliter of the biocompatible solvent which further provides in a PC:DC ratio of between about 1.0:0.80 and about 1.0:1.0 (wt/wt).

Certain embodiments of the inventive PC/DC compositions can provide PC:DC ratios (wt/wt) in narrower ranges within the broader PC:DC ratio range of between 1.0:0.80 and about 1.0:1.0 (wt/wt) above-described and can include PC/DC compositions which provide PC:DC ratios in group including: between about 1.0:0.80 to about 1.0:0.82, between about 1:0:0.81 to about 1.0:0.83, between about 1.0:0.82 and about 1.0:0.84, between about 1.0:0.83 and about 1.0:0.85, between about 1.0:0.84 and about 1.0:0.86, between about 1.0:0.85 and about 1.0:0.87, between about 1.0:0.86 and about 1.0:0.88, between about 1.0:0.87 and about 1.0:0.89, between about 1.0:0.88 and about 1.0:090, between about 1.0:0.89 and about 1.0:0.91, between about 1.0:0.90 and about 1.0:0.92, between about 1.0:0.91 and about 1.0:0.93, between about 1.0:0.92 and about 1.0:0.94, between about 1.0:0.93 and about 1.0:0.95, between about 1.0:0.94 and about 1.0:0.96, between about 1.0:0.95 and about 1.0:0.97, between about 1.0:0.96 and about 1.0:0.98, between about 1.0:0.97 and about 1.0:0.99, between about 1.0:0.98 and about 1.0:1.0, between about 1.0:0.82 and about 1.0:0.83, between about 1:0:0.93 and about 1:0:0.94, between about 1.0:0.96 and about 1.0:0.97, about 1.0:0.83, about 1.0:0.94, and about 1.0:0.97.

A range of embodiments of the inventive PC/DC compositions encompassed by the above-described PC:DC ratios can be achieved by producing a solution which provides an amount of PC per milliliter in the group including: between about 24.0 milligrams and about 25.0 milligrams, between about 24.5 milligrams and about 25.5 milligrams, between about 25.0 milligrams and about 26.0 milligrams, between about 25.5 milligrams and about 26.5 milligrams, between about 26.0 milligrams and about 27.0 milligrams, between about 26.5 milligrams and about 27.5 milligrams, between about 27.0 milligrams and about 28.0 milligrams, between about 27.5 milligrams and about 28.5 milligrams, between about 28.0 milligrams and about 29.0 milligrams, between about 28.5 milligrams and about 29.5 milligrams, between about 29.0 milligrams and about 30.0 milligrams, between about 29.5 milligrams and about 30.5 milligrams, between about 30.0 milligrams and about 31.0 milligrams, between about 30.5 milligrams and about 31.5 milligrams, between about 31.0 milligrams and about 32.0 milligrams, between about 31.5 milligrams and about 32.0 milligrams, between about 24.0 milligrams, about 25.6 milligrams, about 26.4 milligrams, and about 32 milligrams.

Correspondingly, the range of embodiments of the inventive PC/DC compositions encompassed by the above-described PC:DC ratios can be achieved by producing a solution which provides an amount of DC per milliliter in the group including of: between about 19.2 milligrams and about 20.0 milligrams, between about 19.5 milligrams and about 20.5 milligrams, between about 20.0 milligrams and about 21.0 milligrams, between about 20.5 milligrams and about 21.5 milligrams, between about 21.0 milligrams and about 22.0 milligrams, between about 21.5 milligrams and about 22.5 milligrams, between about 22.0 milligrams and about 23.0 milligrams, between about 22.5 milligrams and about 23.5 milligrams, between about 23.0 milligrams and about 24.0 milligrams, between about 23.5 milligrams and about 24.5 milligrams, between about 24.0 milligrams and about 25.0 milligrams, between about 24.5 milligrams and about 25.5 milligrams, between about 25.0 milligrams and about 26.0 milligrams, between about 26.5 milligrams and about 27.5 milligrams, between about 27.5 milligrams and about 28.5 milligrams, between about 28.0 milligrams and about 29.0 milligrams, between about 28.5 milligrams and about 29.5 milligrams, between about 29.0 milligrams and about 30.0 milligrams, between about 29.5 milligrams and about 30.5 milligrams, between about 30.0 milligrams and about 31.0 milligrams, between about 30.5 milligrams and about 31.5 milligrams, between about 31.0 milligrams and about 32.0 milligrams, between about 31.5 milligrams and about 32.0 milligrams, about 19.8 milligrams, about 24.8 milligrams, and about 31.0 milligrams.

A particular embodiment of the inventive PC/DC compositions can provide an amount of phosphatidylcholine of about 24.0 milligrams and an amount of deoxycholate of about 19.8 milligrams per milliliter of the biocompatible solvent establishing a PC:DC ratio of about 1.0:0.083.

Another particular embodiment of the inventive PC/DC compositions can provide an amount of phosphatidylcholine of about 25.6 milligrams and an amount of deoxycholate of about 24.8 milligrams per milliliter of the biocompatible solvent establishing a PC:DC ratio of about 1.0:0.97.

Yet another particular embodiment of the inventive PC/DC compositions can provide an amount of phosphatidylcholine of about 26.4 milligrams and an amount of deoxycholate of about 24.8 milligrams per milliliter of the biocompatible solvent establishing a PC:DC ratio of about 1.0:0.094.

Still another particular embodiment of the inventive PC/DC compositions can provide an amount of phosphatidylcholine of about 32.0 milligrams and an amount of deoxycholate of about 31.0 milligrams per milliliter the biocompatible solvent establishing a PC:DC ratio of about 1.0:0.97.

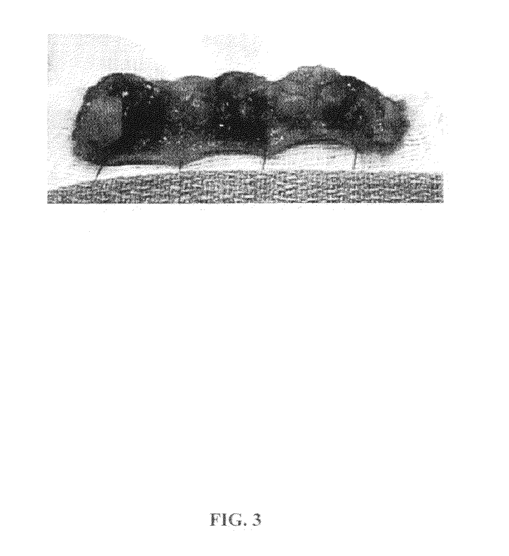

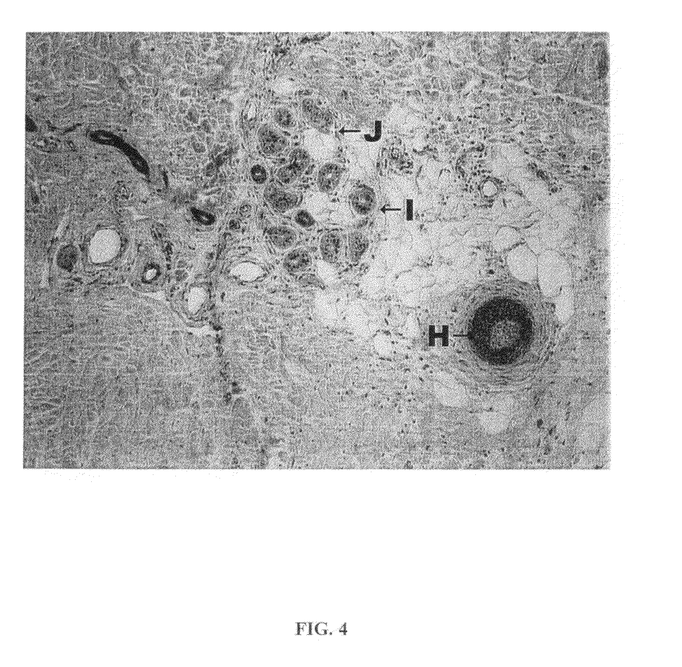

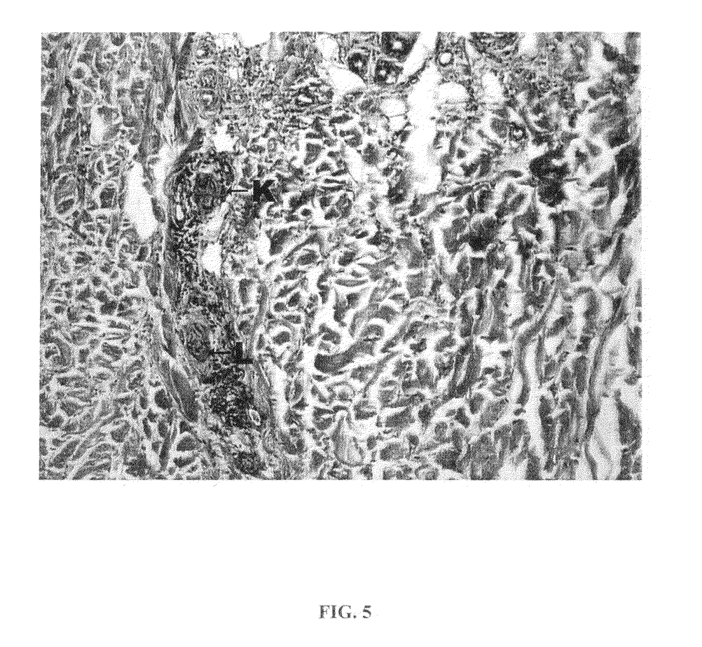

As to each of the above-described PC/DC compositions and other PC/DC compositions which are encompassed by the inventive PC:DC ratios above-described, the inventive PC:DC ratios are between about 1.0:0.80 and about 1.0:1.0 (wt/wt). These inventive PC:DC ratios are based upon the discovery that a PC/DC composition having a PC:DC ratio which reflects an amount of DC of less than about 0.80 (milligrams of DC divided by milligrams of PC) can have a substantially reduced lipolytic action on fat tissue, and that a PC/DC composition which has a PC/DC ratio which reflects an amount of DC of greater than 1.0 (amount of DC in milligrams divided by the amount of PC in milligrams) can be characterized by localized dispersion in the subcutaneous fat layer about the injection location, or a reduced dispersion in the subcutaneous fat layer, or a dispersion that results in substantial “skip areas” between injection sites as shown in FIG. 3 and as above-described, or produces a greater inflammation, necrosis, or bionecrosis of the overlying dermis, eccrine sweat glands, blood vessels, nerve elements, or other tissue element as compared with the inventive PC/DC compositions having the inventive PC/DC ratios.

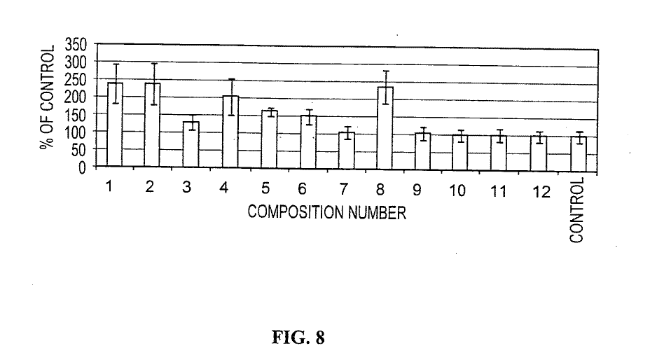

Now referring primarily to FIG. 8, which provides a bar graph that summarizes the results of an in vitro double blind stem cell study which compares the lypolytic action of various DC only formulations (Nos. 4 and 8); PC/DC formulations (Nos. 1, 2, 3, 5, 6); PC only formulations (No. 7); and certain substances added to certain PC/DC formulations (Nos. 9, 10, and 11)(each a “test composition”)(further described in Example 1). Comparison of the results of DC only test composition No. 4 with DC only test composition No. 8 evidences that lypolytic action increases as the concentration of DC in a formulation increases. However, as above described, there can be substantial disadvantages of using DC only formulations as an injectable treatment for fat reduction. However, the addition of PC to a DC only formulation to reduce inflammation, necrosis or bionecrosis and increase dispersion of the DC in tissue, or otherwise offset the disadvantages of using DC only formulation, can result in a reduced the lipolytic action as shown by PC/DC test composition Nos. 3, 5, and 6 which show lesser lipolytic action than DC only test composition Nos. 4 and 8 even though the amount of DC in test composition Nos. 3, 5, and 6 is greater. This is consistent with the results of PC only test composition No. 7 which did not evidence any lipolytic action greater than the control test composition No. 13. While the use of inventive PC/DC compositions having the above described inventive PC:DC ratios and concentrations of PC and DC in solution may be preferable to the use of DC only formulations for injectable treatment for fat reduction, certain inventive PC/DC compositions such as test composition Nos. 1 and 2 can provide even greater lypolytic action using a lesser concentration of PC and a lesser concentration of DC as compared to PC/DC test compositions 3, 5, and 6 when combined with certain additives such as benzyl alcohol, ISUPREL, or Ropivacaine whether separately or in combination each of which separately do not appear to have lipolytic action greater than the control. These inventive embodiments of PC/DC compositions which provide PC:DC ratios of between 1.0:0.80 and 1.0:1.0 and a lesser concentration of DC can be utilized as an injectable (as further described below) for effective fat reduction which reduces or eliminates the disadvantages of using DC only formulations or certain PC/DC formulations.

Any particular embodiment of the PC/DC compositions can further include, without limiting the various biocompatible solvents which can be utilized, an amount of benzyl alcohol of between about 24.0 milligrams per milliliter and about 26.0 milligrams per milliliter.

Any particular embodiment of the PC/DC compositions can further include an amount of anesthetic. Various anesthetics individually or in combination may be included in a particular embodiment of a PC/DC composition based on the application such as: ropivacaine, articaine, benzocaine bupivacaine, chloroprocaine, etidocaine, hexylcaine, lontocaine, lidocatine, levobuivaciaine, mepivacaine, prilocaine, procaine, and tetracaine, it is not intended that the invention be limited by including an anesthetic, or limited to including one or more of the anesthetics described herein. Rather, the anesthetics described herein are intended to provide examples of the numerous and varied anesthetics which may be included in inventive embodiments of the PC/DC compositions depending upon the application. As to particular embodiments of the PC/DC compositions, an amount of ropivacaine of between about 4.0 milligrams per milliliter and about 6.0 milligrams per milliliter can be utilized For example, the particular embodiments of the inventive PC/DC formulation assayed as test compositions Nos. 1 and 2 include an amount of ropivacaine of about 5 mg/mL.

Similarly, any particular embodiment of the PC/DC compositions can further include an amount of beta adrenergic stimulator. A beta adrenergic stimulator can bind either directly or indirectly to the beta-receptor, thereby stimulating it. The stimulated receptor triggers a complex series of events involving multiple enzyme systems which results in an accumulation of cyclic AMP within the cell and decreased ATP. These conditions can activate lipases which break down triglyceride fats in the adipocytes into free fatty acids, which can be used by the cell for growth and metabolism, or may be discharged extracellularly. While various beta adrenergic stimulators individually or in combination can be included in particular embodiment of the PC/DC compositions such as isoproterenol hydrochloride (ISUPREL™), isoproterenol hydrochloride, forskolin, norepinephrine, guarana and clenbuterol, or other beta-receptor specific agonist (or non-specific agonists such as ephedrine as to certain applications) it is not intended that the invention be limited by including any beta adrenergic stimulator, or limited to including a beta adrenergic stimulator described herein. Rather, the beta adrenergic stimulators described herein are intended to provide examples of the numerous and varied beta adrenergic stimulators which can be included in inventive embodiments of the PC/DC compositions. As to particular embodiments of the PC/DC compositions an amount of isoproterenol hydrochloride which provides between about 4 mg/mL and about 10 mg/mL of the PC/DC composition can be utilized. For example, the PC/DC compositions of test composition Nos. 1 and 2 include an amount of isoproterenol hydrochloride which provides 8 mg/mL of the PC/DC composition.

Again, any particular embodiment of the PC/DC compositions can further include, individually or in various permutations or combinations, an amount of collagenase, such as Clostridial collagenase or an amount of one or more of nicotinic acid, clofibrate, tannic acid, scorpion toxin, snake venom, beta adrenic stimulants, dimethlyaminoethanol, hyaluronic acid, penta-O-galloyl-alpha-D-glucose, hormone sensitive lipase, human adipose triglyceride lipase, tnf-alpha, raspberry ketone, ethanol, rosiglitazone, peroxisome-proliferator activated receptor gamma, Y-9738 (ethyl 2(4-chlorophenyl)-5-ethoxy-4-oxazoleacetate) oliphen, fish oil, scallop shell extract, peanut shell extract, and caffeine.

Any of the above-described PC/DC compositions can be provided as part of a kit which includes an amount of PC and an amount of DC in combination, or separately in a manner which allows combination of the amount of PC with the amount of DC, to generate the inventive PC to DC ratios above-described in an amount of biocompatible solvent which can also be provided in the kit or provided separately. Alternately, the kit can provide the amount of PC and the amount of DC already dissolved in the proper amount of biocompatible solvent to generate the inventive PC to DC ratios and concentrations of PC/DC above-described.

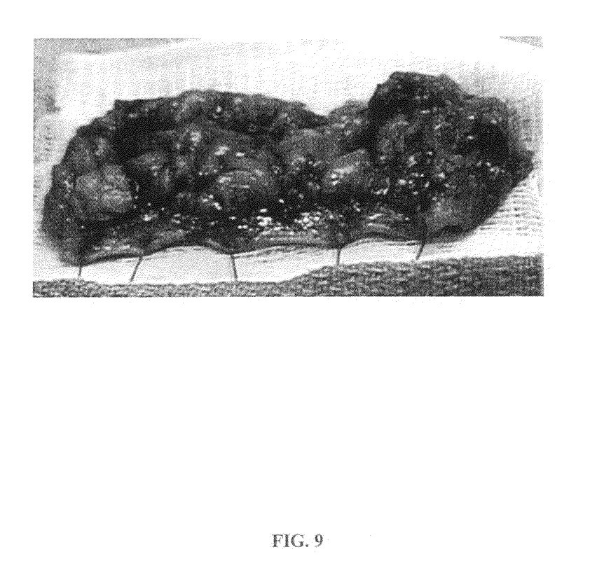

Now referring primarily to FIG. 9, particular embodiments of the inventive PC/DC compositions having a PC:DC ratio of between about 1.0:0.80 and about 1.0:1.0 can provide about 2.6% PC and about 2.5% DC only (as to the particular embodiment of the PC/DC composition utilized in the example shown by FIG. 8 the inventive composition provides PC 26.6 mg/mL and DC 24.8 mg/mL of saline) can be administered 0.5 cc per injection site at a depth of 10 mm with injection sites 1.5 cm apart to result in a pattern of dispersion uninterrupted between injection sites in tissue specimens harvested between about 15 minutes and about one hour post injection.

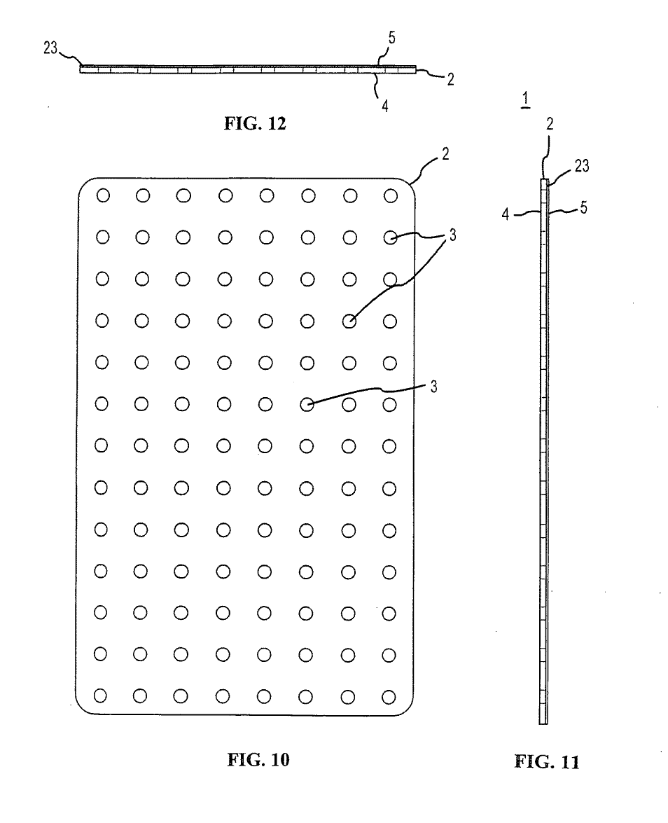

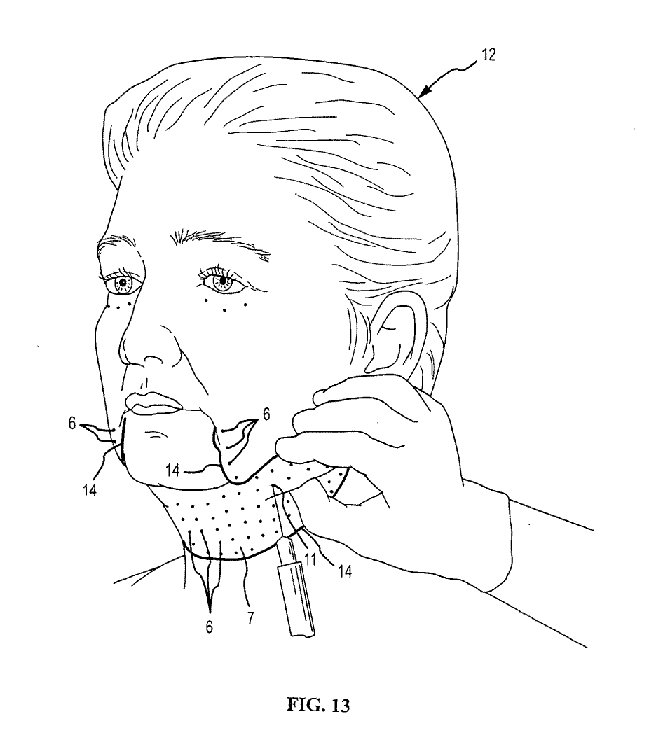

Now referring generally to FIGS. 13-16, while a plurality of injection locations (11) can be established and the PC/DC compositions injected into the fat layer (9) underlying the skin layer (8) without the aid of any additional apparatus, a particular method of administering the PC/DC compositions above-described can in part include the use of an injection location identification template (1), as shown in FIGS. 10-12, which assists in providing a pattern for injection locations (11) which corresponds the dispersion characteristics of a particular embodiment of the PC/DC compositions injected into fat layer (9). As shown by FIGS. 3 and 9, dispersion of DC only compositions in the fat layer (9) can be limited while PC/DC compositions disperse a greater distance in the fat layer (9), the failure to establish the plurality of injection locations (11) in a uniform pattern with the proper distance between each of the plurality of injection locations (11) can result in a pattern of localized cavitation about the sites of injection with areas of untreated uncavitated fat tissue between the injection sites.

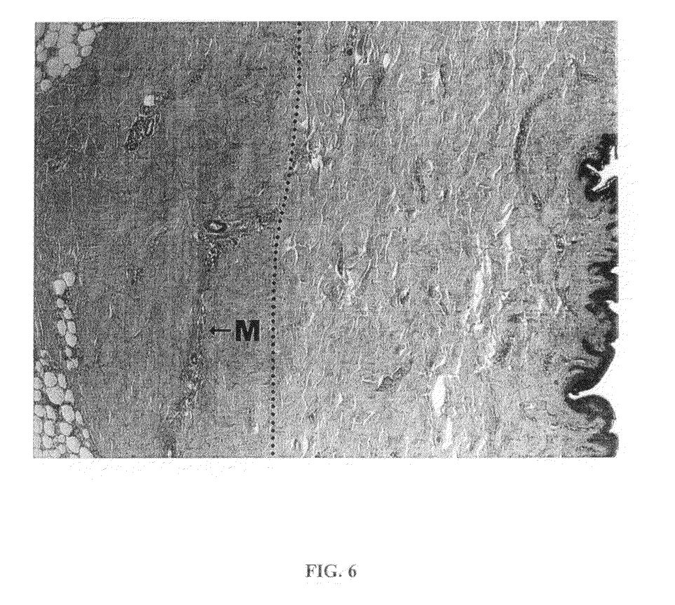

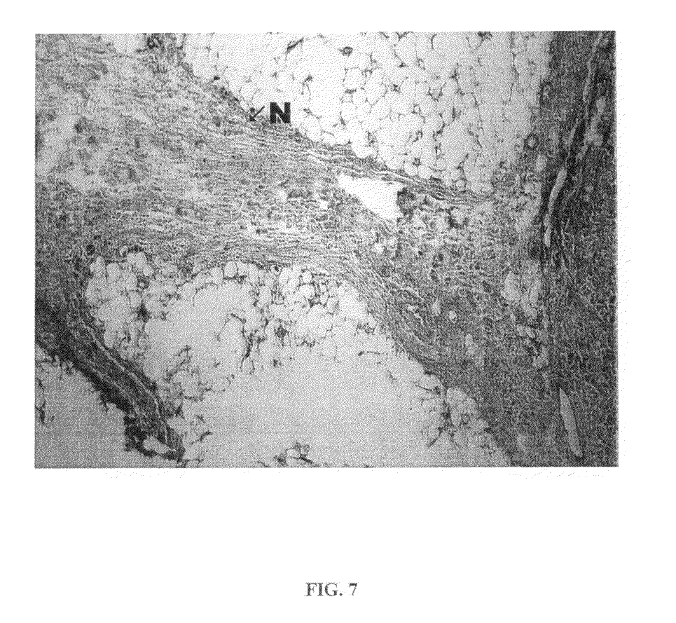

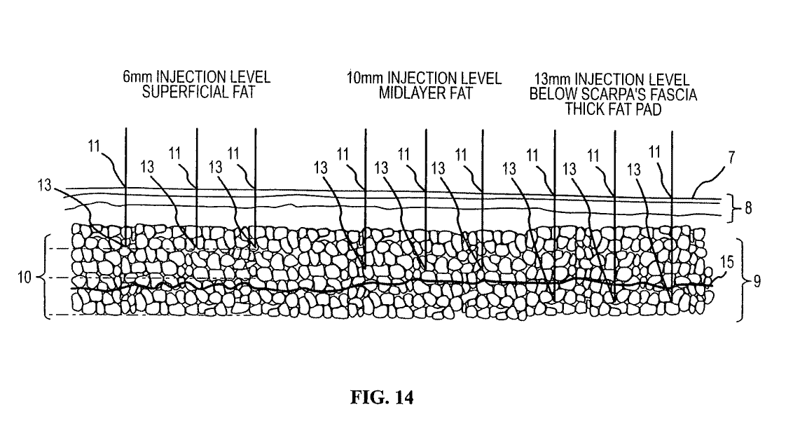

Now referring primarily to FIGS. 10-13, embodiments of the invention can further include an injection location identification template (1) which provides a sheet material (2) having flexure sufficient to allow engagement with a skin layer surface (7) of the skin layer (8) overlying the fat layer (9) (see FIG. 14) to be injectably treated with an embodiment of the PC/DC compositions. The injection location identification template (1) can have a plurality of apertures (3) which communicate between the surface of a first side (4) of the sheet material (2) and the surface of an opposed second side (5) of the sheet material. Each of the plurality of apertures (3) can be configured (for example in the non-limiting circular configuration shown in FIG. 6) to allow a marker (not shown) to pass through the sheet material (2) engaged with a part of the skin layer surface (7) to generate a corresponding plurality of marks (6)(see FIG. 9) on the part of the skin layer surface (7) of the skin layer (8) overlying the fat tissue or fat layer (9) to be injectably treated with any of the embodiments of the PC/DC compositions. Each of the plurality of marks (6) generated on the part of the skin layer surface (7) utilizing the injection location identification template (1) are viewable upon disengaging the injection location identification template (1) from the skin layer surface (7). Each of the plurality of marks (6) identifies an injection location (11) at which the PC/DC composition (also referred to as the “injectable composition”) can be injected by use of an injection needle or other injector to establish an amount of the PC/DC composition at one or more of a plurality of levels (10) in the fat layer (9) beneath the skin layer (8).

Numerous and varied materials can be utilized as the sheet material from which embodiments of the injection location identification template (1) so long as the sheet material has flexibility to conform sufficiently to the part of the skin layer surface (7) to allow a corresponding plurality of marks (6) to be established on the skin layer surface (7). For example the sheet material can without limitation be a plastic sheet material, a cloth sheet material, a web material, or the like in which the plurality of apertures (3) can be established. The sheet material (2) can further include an adhesive layer (23) which allows releasably fixed engagement of the sheet material (2) with the skin layer surface (7). Each of the plurality of apertures (3) of a particular injection location identification template (1) can be established a uniform distance apart of between about 0.7 cm and 1.5 cm to generate a rectilinear pattern as shown for example in FIG. 13. The injection location identification template (1) can have any manner of perimeter configuration and it is not intended that the perimeter configuration shown in FIG. 10 be limiting with respect to manner in which the injection location template can be configured.

Now referring primarily to FIG. 13, the inventive PC/DC compositions can be administered by locating at least one injection location (11) on a skin layer surface (7) (such as a location on the surface of the skin of a person (12)) at which the injection needle can be inserted through the skin layer (8) to establish an amount of the PC/DC composition at a level in the underlying fat tissue (9) (also referred to herein as a “fat layer”)(see FIG. 10). The term “underlying” encompasses that part of a fat tissue or a fat layer (9) located beneath the skin layer (8) injectable from a particular an injection location (11) regardless of the type of injection needle or the method of injection utilized. The term “a level” means the depth at which the PC/DC composition is established in the fat layer (9). For example, a 6 milliliter (“mm”) level means that the injection needle tip (13) has a position in the fat layer which can establish the PC/DC composition at a depth of about 6 mm into the fat layer. Naturally, in practice a plurality of injections of a PC/DC composition at a corresponding plurality of injection locations (11) intended to establish the PC/DC composition at one uniform level (10) may include an amount of variability in the actual level at which the injected amount of PC/DC composition is established depending on the injector, the type of injection needle utilized, and so forth. A reasonable variation in the level (10) between a plurality of injection locations (11) or persons (12) treated is to be understood as encompassed in the definition of any particular level (10) in the fat layer (9).

Now referring primarily to FIGS. 10-13, a person (12) may make preliminary markings (14) around the areas they want treated with the PC/DC compositions. The injection location identification template (1) may be used to generate the plurality of marks (6) on the skin layer surface (7) each of which indicates one of the plurality of injection locations (11) as shown in FIG. 13. For example, a injection location template (1) having a plurality of apertures (3) spaced uniformly about 1.5 cm apart can utilized in those instances in which the fat layer (9) to be treated underlies a relatively large area of the skin layer (8). An injection location identification template (1) having a plurality of apertures (3) spaced uniformly 1.0 cm apart can be used to treat fat layers (9) having a smaller area of comparably dense fat tissue. When treating cellulite or other skin deformities, an injection location identification template (1) having a plurality of apertures (3) spaced 0.7 cm apart can be utilized.

Now referring primarily to FIG. 14, the level (10) at which the PC/DC composition is established in the fat layer (9) can vary according to the application of the PC/DC compositions. With respect to PC/DC compositions used for lipolysis (fat reduction), if the fat layer (9) is thick, a 13-mm 20 to 32 gauge needle may be used to inject about 0.4 mL of the PC/DC composition per each of the plurality of injection locations (11) spaced about 1.5 cm apart to establish the PC/DC composition at a level (10) of about 13 mm which can be below the scarpa's facia (15). Typically, for the purpose of lipolysis a midlevel subcutaneous 0.4 mL injection of the PC/DC composition at a level (10) of about 10 mm into the fat layer (9) can be utilized. While the examples above-described and shown in FIG. 10 set out particular levels (10) in the fat layer (9), injection volume, and spacing of the plurality of injection locations (11), this is not intended to be limiting with respect to establishing the PC/DC composition at other levels in the fat layer (9), using alternate PC/DC composition injection volumes, or using alternate spacing for the plurality of injection locations (11).

Rather, the examples are intended to be illustrative of a wide range of levels which at which the PC/DC compositions can be established in the fat layer (9) depending upon the particular application. In general, establishing the PC/DC composition at a level (10) in the middle of the fat layer (9) can confer an advantage as to fat reduction, while establishing the PC/DC at a level in the upper third of the fat layer (9) can confer an advantage as smoothing the skin layer (8). Depending on the application, one or more than one level (10) may be selected for the same region treated with the PC/DC compositions. Similarly, uniform spacing as opposed to non-uniform spacing of the plurality of injection locations (11) can confer an advantage in using the PC/DC compositions.

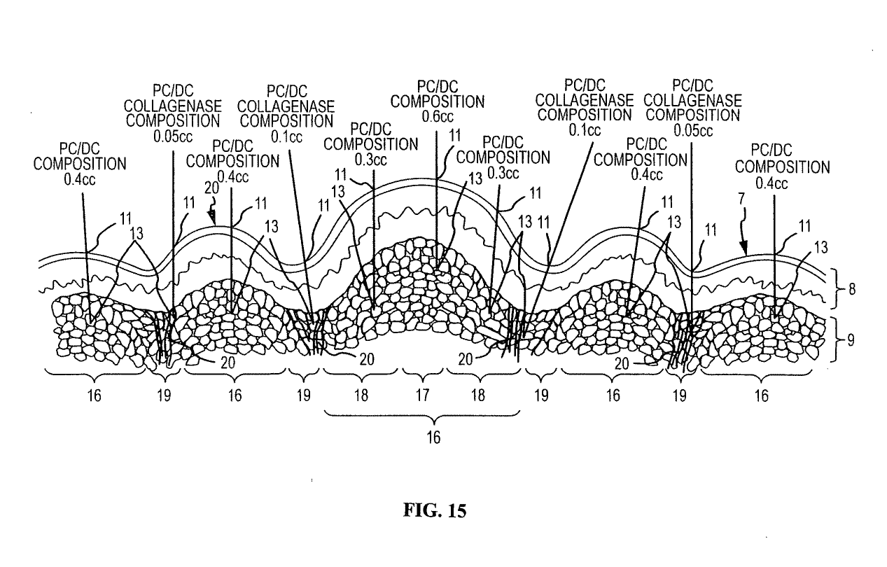

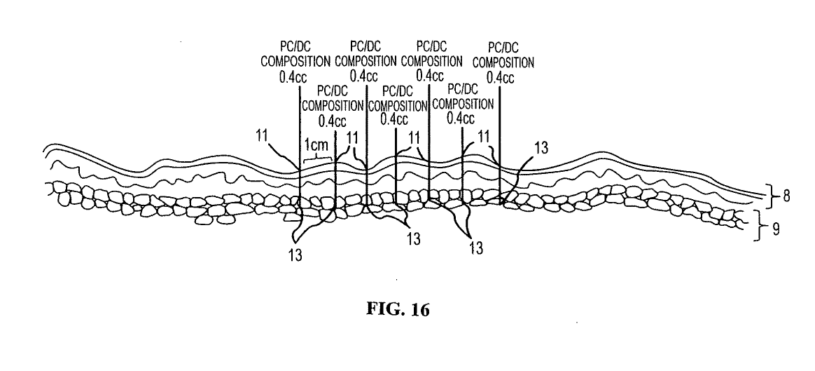

Now referring primarily to FIG. 15, an inventive method of cellulite treatment can include a treatment of the thicker areas of fat tissue (sometimes referred to as “hills” or “lumps”)(16) with the PC/DC compositions. The “hills” (16) to be treated can be identified by marking the skin surface (typically circles about the hill or lump) with addition circling of areas of the hill that are protuberant. The circles within the circles denote a thicker area of fat tissue (17) which can be treated with greater injection volume of the PC/DC composition such as about 0.6 cc per injection location (11), and the peripheral circles where thickness of the fat tissue tapers down (18) can be injected with a decreased injection volume of the PC/DC composition such as about 0.1 cc to about 0.3 cc per injection location (11). An injection location identification template (1)(or other grid or injection location identification technique) can be used to locate a plurality of injection locations (11) about 1.2 cm apart on the surface of the hill (16). Injection of the PC/DC composition at each injection location (11) at a depth of about 10 mm can be accomplished with an injection needle or mesogun. In certain embodiments of the inventive method of cellulite treatment, only the “hills” (16) are treated during the first session.

Again referring primarily to FIG. 15, an inventive method of cellulite treatment can include a treatment of the thinner areas or depressions (19) in the fat layer (9)(sometime referred to as “divots” or “valleys”). The divots (19) can be identified by marking the skin layer surface (7). The depth of the divot (19) can be correlated with the dose of a PC/DC-collagenase composition. Specifically with respect to embodiments of the PC/DC composition useful in treating divots (19), collagenase derived from Clostridium endotoxin can be further included by combining equal parts of a collagenase solution of about 250 units/mL and a PC/DC composition (the “PC/DC-collagenase composition”). A certain non-limiting embodiment of the PC/DC-collagenase composition provides a concentration of 4.5% PC and 4.2% DC in the biocompatible solvent.

The PPC-collagenase mixture can be injected at a volume of between about 0.05 cc to about 0.5 cc into the fat layer (9) underlying the divot (9) in the skin layer (8). Divots (19) of greater depth can be injected with a volume of about 0.3 cc to about 0.5 cc of the PC/DC-collagenase composition while divots (19) of lesser depth can be injected with a volume of about 0.05 cc to about 0.25 cc of the PC/DC-collagenase composition. The PC/DC-collagenase composition injection sites (11) can be about 0.5 cm apart. Injection of the PC/DC-collagenase composition can be performed with a 26 gauge ⅜″ needle which can be held at about a 45 degree angle to the divot (19) in order to better address the fibrous tissue (20). The PC/DC-collagenase composition injection location (11) can be established at the base of the divot (17), or if the divot comprises a broader depression, the injection locations (11) can be established about 0.5 cm apart.

Now referring primarily to FIG. 14, if skin retraction or smoothing of the skin layer (8) is the primary goal of injecting the PC/DC compositions, a 6-mm needle may be used to inject about 0.4 mL of the PC/DC composition per each of the plurality of injection locations (11) spaced about a 1-cm apart to establish the PC/DC composition at a level of about 6 mm into the fat layer (9).

### EXAMPLE 1

### In Vitro Double Blind Stem Cell Study of Lipolytic Formulas

Lipolysis induced in cultured human adipocytes by certain embodiments of the inventive injectable compositions for fat reduction and their constituent components was assayed in vitro under controlled conditions. The results show that compositions for fat reduction which include phosphatidylcholine-deoxycholate perform optimally within a narrow range of PC:DC ratios.

Cells utilized for the in vitro assays were obtained from five different dermolipectomy specimens. Preadipocytes were cultured in differentiation media over 14 days to produce adipocytes incubated between 12-14 hours in each of 12 test solutions. Each of the cell lines incubated in each of the 12 test solutions were split and introduced into four in vitro assays.

Cell lines were incubated in each of the following compositions:
1. Phosphatidylcholine 25.6 milligrams per milliliter (mg/mL)/Deoxycholate 24.8 mg/mL, Benzyl Alcohol 25.6 mg/mL, ISUPREL 8 mg/mL, and Ropivacaine 5 mg/mL.
2. Phosphatidylcholine 24.0 mg/mL/Deoxycholate 19.8 mg/mL, Benzyl Alcohol 25.6 mg/mL, ISUPREL 8 mg/mL, and Ropivacaine 5 mg/mL.
3. Phosphatidylcholine 50.0 mg/mL/Deoxycholate 42.0 mg/mL.
4. Deoxycholate 10.0 mg/mL.
5. Phosphatidylcholine 45.0 mg/mL/Deoxycholate 42.0 mg/mL.
6. Phosphatidylcholine 53.0 mg/mL/Deoxycholate 42.0 mg/mL.
7. Phosphatidylcholine 50 mg/mL in mineral oil.
8. Deoxycholate 24.8 mg/mL.
9. Benzyl alcohol 25.6 mg/mL.
10. ISUPREL 8.0 mg/mL.
11. Ropivacaine 5.0 mg/mL.
12. Vitamin B complex and pentoxyffline
13. Control--Saline Solution.

Each of the cell lines incubated in the above-described test compositions were split and introduced into each of four assays, as follow:

A. Lactate Dehydrogenase Detection (“LDH”). Cell death and cell lysis was quantitated by measurement of lactate dehydrogenase activity corresponding to cytosol released from lysed cells into the supernatant using a Roche LDH Assay, Catalog No. 11644793001. The supernatant was collected cell-free and then incubated with the substrate mixture provided in the Roche LDH Assay kit. LDH activity was determined by a coupled enzymatic reaction whereby tetrazolium salt INT is quantitatively reduced to formazan based on the level of LDH activity. Formazan is a water soluble dye having an absorption maximum at approximately 500 nanometers.

B. Basal Lipolysis Rate. Free lipid in collected supernatant was quantified using a Lipolyis assay available from Zan-Bio, Catalog No. LIP-1 to measure released glycerol. Glycerol released to the supernatant is phosphorylated by adenosine triphosphate to form glycerol-1-phosphate and adenosine-5′-diphosphate in a reaction catalyzed by glycerol kinase. Glycerol-1-phosphate is then oxidized by glycerol phosphate oxidate dihydroxyacetone phosphate and hydrogen peroxide. A quinoneimine dye is produced by the peroxidase catalyzed coupling of 4-aminoanitpyrine and sodium N-ethyl-N-(3-sulfopropyl) manisidine with hydrogen peroxide. The quinoneimine dye has an absorbance maximum at 540 nanometers. The increase in absorbance at 540 nanometers is directly proportionate to glycerol concentration in the supernatant.

C. Surface Area of Lipid Laden Cells (“Oil Red O”). Cell lines were seeded into 10 cm Petri dishes and differentiated for two weeks. The differentiated cells in four experimental plates were incubated with each test composition and the control for 12 hours. Subsequent to incubation with the test compositions and the control the incubated cells were stained with oil red O specific for lipid. The surface area of lipid laden cells was then quantified using imaging software obtained from the National Institutes of Health.

D. Triglyceride Detection. The amount of triglyceride was quantified by measurement of glycerol released by cell lysis into the supernatant utilizing a triglyceride assay kit available from Zen-Bio, Catalog No. TG-1-NC. There is a molar equivalent of triglyceride for each mole of glycerol detected.

The results are summarized in the bar graph of FIG. 8. In general, test composition nos. 1, 2, 4, and 8 generated a detectable increase in free lipids over the control in both lipolysis assays. The concentration of deoxycholate correlates with increased lipolysis. The addition of phosphatidylcholine to deoxycholate evidences a narrow range of peak performance ratios. Test composition nos. 3, 5, and 6 showed significant cytotoxicity in the Oil Red O and LDH assays. Not surprisingly, isolated phosphatidylcholine (test composition no. 7) showed no significant lipolytic or cytotoxic activity when compared to the saline control. Because this molecule is a major component of all cell membranes and has a neutral pH of 7.0, it would be unlikely that PC would be able to lyse the cell that it is supposed to protect. None of the “enhancement” additives 9-12 evidenced any statistically significant lipolytic properties at the concentrations assayed.

Specifically, the results show that test composition no. 1 (phosphatidylcholine 25.6 milligrams per milliliter (mg/mL)/deoxycholate 24.8 mg/mL) can be a potent lipolytic injectable equally as effective as deoxycholate 24.8 mg/mL but without the disadvantages above discussed associated with utilizing DC only. The amount of PC is not so great as to reduce the effect of DC, yet there is enough PC present to allow dispersion of the composition in the fat tissue. The PC:DC ratio is about 1.0:0.97.

Test composition no. 2 (phosphatidylcholine 24.0 mg/mL/deoxycholate 19.8 mg/mL) can also be a potent lipolytic injectable. There appears to be no statistically significant difference in lipolytic activity between composition no. 1, composition no. 2, and test composition no. 9; however, use of test composition no. 2 may be preferable because it contains PC in an amount which provides a PC:DC ratio of about 1.0:0.83 which avoids many of the disadvantages of deoxycholate only of test composition no. 9.

Test composition no. 3 (phosphatidylcholine 50 mg/ml and deoxycholate 42 mg/ml) is a conventional PC-DC fat reduction composition but evidences significantly lower lypolytic activity than embodiments of the inventive fat reduction composition nos. 1 and 2.

Test composition no 4 (deoxycholate 10 mg/ml) appears to be less lipolytic than either of test composition nos. 1 or 2 based on the average of n=20. While phosphatidylcholine is not inherently lipolytic, the synergistic combination of test composition nos. 1 and 2 appears to be more effective at lysing fat cells than DC only formulations.

Test composition no. 5 (phosphatidylcholine 45.0 mg/mL/deoxycholate 42.0 mg/mL) performed slightly better than test composition no. 3 (phosphatidylcholine 50.0 mg/mL/deoxycholate 42.0 mg/mL) in the lipolysis/cytotoxicity assays. These results evidence that withdrawal of PC, up to a certain point, can increase lipolytic action of a PC/DC composition. While the ratio of test composition no. 5 (PC:DC 1.0:0.93) fits within the inventive PC:DC ratio range provides a greater concentration of both PC and DC than test composition nos. 1 and 2, unexpectedly the lipolytic action of test composition no. 5 is lesser than either of test composition nos. 1 or 2.

Test composition no. 6 (phosphatidylcholine 53.0 mg/mL/deoxycholate 42.0 mg/mL) appears to be slightly less lipolytic than test composition no. 5 (Phosphatidylcholine 45.0 mg/mL/Deoxycholate 42.0 mg/mL), but appears to be somewhat more lipolytic than test composition no. 3 (phosphatidylcholine 50.0 mg/mL/deoxycholate 42.0 mg/mL).

Test composition no. 7 (phosphatidylcholine 50 mg/mL in mineral oil). To date, it is believed there have been no reports of successful isolation of PC from DC. A mineral oil solution was found that was also tested individually (without PC) in order to confirm the absence of lipolytic or cytotoxic activity. The phosphatidylcholine solution did not appear to cause any significant lipolysis; it rated the same as the control. This finding confirms that while PC is an important part of the drug delivery mechanism and also acts as a buffer, it does not directly cause lipolysis.

Test composition no. 8 (deoxycholate 24.8 mg/mL) By withdrawing all phosphatidylcholine, we can see the degree of suppression of lipolytic action that PC can causes. Unexpectedly, the degree of lipolysis of test composition no. 8 was only slightly greater than test composition no. 3 (though the histological differences are quite profound in vivo) and was less than the level of lipolysis obtained test composition nos. 1 and 2.

Test composition no. 9 (benzyl alcohol 25.6 mg/mL). Benzyl alcohol has been thought to cause lipolysis for many years; however, at the concentration used in test composition nos. 1 and 2 it does not appear to induce lipolysis or cytotoxic damage and individually does not appear to have any lipolytic action greater than the control.

Test composition no. 10 (ISUPREL 8.0 mg/mL). Beta-adrenergic drugs are widely used to induce lipolysis. While ISUPREL and epinephrine are considered lipolytic, the lipolysis is not “permanent” as the cell membrane does not rupture. This study evidences that ISUPREL does not cause cell wall disruption or cytotoxicity.

Test composition no. 11 (Ropivacaine 5.0 mg/mL) Ropivacaine is a long lasting local anesthetic used in test composition nos. 1 and 2 and can reduce pain on injection and can last from 30 minutes to 2 hours post-injection. The study appears to confirm that ropivacaine does not cause lipolysis or cytotoxicity.

Test composition no. 12 (Vitamin B complex and pentoxyffline). The B-vitamin complex and vasodilator pentoxyffline does not appear to cause lipolysis or cytotoxicity.

As can be easily understood from the foregoing, the basic concepts of the present invention may be embodied in a variety of ways. The invention involves numerous and varied embodiments of PC/DC compositions and methods of using such embodiments of the PC/DC compositions for the reduction of fat. This International Cooperation Treaty Application claims the benefit of U.S. Provisional Patent Application No. 60/830,947, filed Jul. 14, 2006, and U.S. Provisional Patent Application No. 60/860,838, filed Nov. 22, 2006, the entirety of each application including the description along with any photographs, figures, or tables is hereby incorporated by reference herein.

As such, the particular embodiments or elements of the invention disclosed by the description or shown in the figures or tables accompanying this application are not intended to be limiting, but rather exemplary of the numerous and varied embodiments generically encompassed by the invention or any particular part or element of the invention or equivalents thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of “injectable compositions” should be understood to encompass disclosure of the act of “injecting compositions”—whether explicitly discussed or not—and, conversely, were there effectively disclosure of the act of “injecting compositions”, such a disclosure should be understood to encompass disclosure of “injectable compositions” and even a “means for injecting compositions.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the PC/DC compositions disclosed and described herein, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally any claims set forth in this specification are intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1-28. (canceled)

29. A method of administering an injectable composition for fat reduction, comprising the steps of:
a) generating said injectable composition as a solution which provides an amount of phosphatidylcholine and an amount of deoxycholate in an amount of biocompatible solvent in a ratio of between about 1.0:0.80 and about 1.0:1.0;
b) locating at least one injection location on a skin layer surface;
c) inserting an injection needle through a skin layer at said at least one injection location to locate an injection needle tip at a level in a fat layer beneath said skin layer; and
d) establishing an amount of said injectable composition for fat reduction at said level in said fat layer beneath said skin layer.

30. The method of administering an injectable composition for fat reduction of claim 29, wherein said step of generating said injectable composition as a solution which provides an amount of phosphatidylcholine and an amount of deoxycholate in an amount of biocompatible solvent in a ratio of between about 1.0:0.80 and about 1.0:1.0 further comprises the step of providing said amount of phosphatidycholine in said solution at between about 24 milligrams per milliliter and about 32 milligrams per milliliter.

31. The method of administering an injectable composition for fat reduction of claim 30, wherein said step of generating said injectable composition as a solution which provides an amount of phosphatidylcholine and an amount of deoxycholate in an amount of biocompatible solvent in a ratio of between about 1.0:0.80 and about 1.0:1.0 further comprises the step of providing said amount of deoxycholate in said solution at between about 19.2 milligrams per milliliter and about 32 milligrams per milliliter.

32. The method of administering an injectable composition for fat reduction of claim 31, wherein said step of generating said injectable composition as a solution which provides an amount of phosphatidylcholine and an amount of deoxycholate in an amount of biocompatible solvent in a ratio of between about 1.0:0.80 and about 1.0:1.0 further comprises the step of providing said amount of phophatidylcholine and said amount of deoxycholate in said solution in a ratio selected from the group consisting of: between about 1.0:0.80 to about 1.0:0.82, between about 1:0:0.81 to about 1.0:0.83, between about 1.0:0.82 and about 1.0:0.84, between about 1.0:0.83 and about 1.0:0.85, between about 1.0:0.84 and about 1.0:0.86, between about 1.0:0.85 and about 1.0:0.87, between about 1.0:0.86 and about 1.0:0.88, between about 1.0:0.87 and about 1.0:0.89, between about 1.0:0.88 and about 1.0:090, between about 1.0:0.89 and about 1.0:0.91, between about 1.0:0.90 and about 1.0:0.92, between about 1.0:0.91 and about 1.0:0.93, between about 1.0:0.92 and about 1.0:0.94, between about 1.0:0.93 and about 1.0:0.95, between about 1.0:0.94 and about 1.0:0.96, between about 1.0:0.95 and about 1.0:0.97, between about 1.0:0.96 and about 1.0:0.98, between about 1.0:0.97 and about 1.0:0.99, between about 1.0:0.98 and about 1.0:1.0, between about 1.0:0.82 and about 1.0:0.83, between about 1:0:0.93 and about 1:0:0.94, between about 1.0:0.96 and about 1.0:0.97, about 1.0:0.83, about 1.0:0.94, and about 1.0:0.97.

33. The method of administering an injectable composition for fat reduction of claim 30, wherein said step of providing said amount of phosphatidycholine in said solution at between about 24 milligrams per milliliter and about 32 milligrams per milliliter comprises the step of providing an amount of phosphatidylcholine selected from the group consisting of: between about 24.0 milligrams and about 25.0 milligrams, about 24.5 milligrams and about 25.5 milligrams, about 25.0 milligrams and about 26.0 milligrams, about 25.5 milligrams and about 26.5 milligrams, about 26.0 milligrams and about 27.0 milligrams, about 26.5 milligrams and about 27.5 milligrams, about 27.0 milligrams and about 28.0 milligrams, about 27.5 milligrams and about 28.5 milligrams, about 28.0 milligrams and about 29.0 milligrams, about 28.5 milligrams and about 29.5 milligrams, about 29.0 milligrams and about 30.0 milligrams, about 29.5 milligrams and about 30.5 milligrams, about 30.0 milligrams and about 31.0 milligrams, about 30.5 milligrams and about 31.5 milligrams, about 31.0 milligrams and about 32.0 milligrams, about 31.5 milligrams and about 32.0 milligrams, about 24.0 milligrams, about 25.6 milligrams, about 26.4 milligrams, about 32 milligrams.

34. The method of administering an injectable composition for fat reduction of claim 31, wherein said step of providing said amount of deoxycholate in said solution at between about 19.2 milligrams per milliliter and about 32 milligrams per milliliter comprises the step of comprises the step of providing an amount of deoxycholate selected from the group consisting of: between about 19.2 milligrams and about 20.0 milligrams, 19.5 milligrams and about 20.5 milligrams, 20.0 milligrams and about 21.0 milligrams, 20.5 milligrams and about 21.5 milligrams, 21.0 milligrams and about 22.0 milligrams, 21.5 milligrams and about 22.5 milligrams, 22.0 milligrams and about 23.0 milligrams, 22.5 milligrams and about 23.5 milligrams, 23.0 milligrams and about 24.0 milligrams, 23.5 milligrams and about 24.5 milligrams, 24.0 milligrams and about 25.0 milligrams, 24.5 milligrams and about 25.5 milligrams, 25.0 milligrams and about 26.0 milligrams, 26.5 milligrams and about 27.5 milligrams, 27.5 milligrams and about 28.5 milligrams, 28.0 milligrams and about 29.0 milligrams, 28.5 milligrams and about 29.5 milligrams, 29.0 milligrams and about 30.0 milligrams, 29.5 milligrams and about 30.5 milligrams, 30.0 milligrams and about 31.0 milligrams, 30.5 milligrams and about 31.5 milligrams, 31.0 milligrams and about 32.0 milligrams, 31.5 milligrams and about 32.0 milligrams, about 19.8 milligrams, about 24.8 milligrams, about 31.0 milligrams.

35. The method of administering an injectable composition for fat reduction of claim 30, wherein said step of generating said injectable composition as a solution which provides an amount of phosphatidylcholine and an amount of deoxycholate in an amount of biocompatible solvent in a ratio of between about 1.0:0.80 and about 1.0:1.0 comprises the step of generating said injectable composition as a solution which provides an amount of phosphatidylcholine of about 24.0 milligrams per milliliter and an amount of deoxycholate of about 19.8 milligrams per milliliter of said amount of biocompatible solvent in a ratio of between about about 1.0:0.83.

36. The method of administering an injectable composition for fat reduction of claim 30, wherein said step of generating said injectable composition as a solution which provides an amount of phosphatidylcholine and an amount of deoxycholate in an amount of biocompatible solvent in a ratio of between about 1.0:0.80 and about 1.0:1.0 comprises the step of generating said injectable composition as a solution which provides an amount of phosphatidylcholine of about 25.6 milligrams per milliliter and said amount of deoxycholate of about 24.8 milligrams per milliliter having a ratio of about 1.0:0.97.

37. The method of administering an injectable composition for fat reduction of claim 30, wherein said step of generating said injectable composition as a solution which provides an amount of phosphatidylcholine and an amount of deoxycholate in an amount of biocompatible solvent in a ratio of between about 1.0:0.80 and about 1.0:1.0 comprises the step of generating said injectable composition as a solution which provides an amount of phosphatidylcholine of about 26.4 milligrams per milliliter and said amount of deoxycholate of about 24.8 milligrams per milliliter having a ratio of about 1.0:0.94.

38. The method of administering an injectable composition for fat reduction of claim 30, wherein said step of generating said injectable composition as a solution which provides an amount of phosphatidylcholine and an amount of deoxycholate in an amount of biocompatible solvent in a ratio of between about 1.0:0.80 and about 1.0:1.0 comprises the step of generating said injectable composition as a solution which provides an amount of phosphatidylcholine of about 32.0 milligrams per milliliter and said amount of deoxycholate of about 31.0 milligrams per milliliter having a ratio of about 1.0:0.97.

39. The method of administering an injectable composition for fat reduction of claim 31, further comprising the step of admixing in said solution an amount of benzyl alcohol of between about 24.0 milligrams per milliliter and about 26.0 milligrams per milliliter.

40. The method of administering an injectable composition for fat reduction of claim 39, further comprising the step of admixing in said solution an amount of ISUPREL of between about 7.0 milligrams per milliliter and about 9.0 milligrams per milliliter.

41. The method of administering an injectable composition for fat reduction of claim 39, further comprising the step of admixing in said solution an amount of ropivacaine of between about 4.0 milligrams per milliliter and about 6.0 milligrams per milliliter

42. The method of administering an injectable composition for fat reduction of claim 41, further comprising the steps of:
a) admixing in said solution an amount of benzyl alcohol of between about 24.0 milligrams per milliliter and about 26.0 milligrams per milliliter;
b) admixing in said solution an amount of ISUPREL of between about 7.0 milligrams per milliliter and about 9.0 milligrams per milliliter; and
c) admixing in said solution providing an amount of ropivacaine of between about 4.0 milligrams per milliliter and about 6.0 milligrams per milliliter.

43. The method of administering an injectable composition for fat reduction of claim 29, wherein said step of locating said at least one injection location on said skin layer surface comprises the step of locating a plurality of injection locations a distance apart of between about 0.75 centimeters to about 2.0 centimeters.

44. The method of administering an injectable composition for fat reduction of claim 43, wherein said step of locating said at least one injection location on said skin layer surface comprises the step of locating a plurality of injection locations a distance apart of between about 1.0 centimeter to about 1.5 centimeters.

45. The method of administering an injectable composition for fat reduction of claim 43, wherein said step of establishing said amount of said injectable composition for fat reduction at said level in said fat layer beneath said skin layer comprises the step of establishing said amount of said injectable composition for fat reduction at a middle level in said fat layer beneath said skin layer.

46. The method of administering an injectable composition for fat reduction of claim 45, wherein said step of establishing said amount of said injectable composition at a middle level in said fat layer beneath said skin layer comprises the step of establishing said amount of said injectable composition for fat reduction at a level in said fat layer beneath said skin layer of between about 10 millimeters and about 13 millimeters.

47. The method of administering an injectable composition for fat reduction of claim 43, wherein said step of establishing said amount of said injectable composition at a level in a fat layer beneath said skin layer comprises the step of establishing said amount of said injectable composition for fat reduction at a upper level in said fat layer beneath said skin layer.

48. The method of administering an injectable composition for fat reduction of claim 47, wherein said step of establishing said amount of said injectable composition at a upper level in said fat layer beneath said skin layer comprises the step of establishing said amount of said injectable composition at a level in said fat layer beneath said skin layer of between about 6 millimeters and about 10 millimeters.

49. The method of administering an injectable composition for fat reduction of claim 48, further comprising the step of generating an increased skin retraction of said skin layer as said level of said injectable composition for fat reduction established in said fat layer approaches about 6 millimeters.
